(19) United States
(12) Patent Application Publication
(10) Pub. No.: US 2022/0273738 A1
(43) Pub. Date: Sep. 1, 2022

(54) ONCOLYTIC VIRUS AND APPLICATION THEREOF, AND DRUG FOR TREATING CANCER

(71) Applicant: WU, Zetang, Hefei, Anhui (CN)

(72) Inventor: Zetang WU, Hefei, Anhui (CN)

(21) Appl. No.: 17/597,653

(22) PCT Filed: Jun. 5, 2020
(86) PCT No.: PCT/CN2020/094751

§ 371 Date: Jan. 15, 2022

(30) Foreign Application Priority Data
Jul. 16, 2019 (CN) 201910642804.4

(51) Int. Cl. A61K 35/763; A61P 35/00; C12N 15/86; C12N 7/00; C12N 9/50

(52) U.S. Cl. CPC A61K 35/763; A61P 35/00; C12N 15/86; C12N 7/00; C12N 9/506; C12N 2710/16632; C12N 2710/16643; C12N 2830/008

(57) ABSTRACT

Provided are an oncolytic virus and an application thereof, and a drug for treating a cancer. A first regulatory element and a second regulatory element are inserted into the genome of the oncolytic virus. The first regulatory element comprises a tumor-specific promoter and a first nucleic acid sequence, which is driven by the cancer cell specific promoter to express a specific protease in tumor cells; the second regulatory element comprises a second nucleic acid sequence for encoding an extracellular secretion signal peptide and a third nucleic acid sequence for encoding a specific cleavage site. The oncolytic virus can be replicated in tumor cells effectively to kill tumor cells while being safe to non-cancer cells.

### CROSS-REFERENCES TO RELATED APPLICATIONS

The present disclosure claims the priority of a Chinese patent application filed with the Chinese Patent Office on Jul. 16, 2019, with the application number 2019106428044 and entitled “Oncolytic Virus and Application Thereof and Drug for Treating Cancer”, the entire content of which is incorporated by reference in this application.

### TECHNICAL FIELD

The present disclosure relates to the technical field of cancer treatment, in particular, to an oncolytic virus and use thereof and drug for treating cancer.

### BACKGROUND ART

Cancer has become the number one killer affecting health. China is a region with a high incidence of cancer, especially lung cancer, gastric cancer, liver cancer and rectal cancer. According to statistics, in 2016 alone, there were 4.8 million new cancer patients nationwide, and 2.3 million patients died of various cancers. With the advancement of technology, various new treatments, especially immunotherapy, have been continuously put into clinical use. However, the demands for safe and effective therapies are far from being met. Therefore, it is imperative to develop new drugs for the treatment.

An article published in “Science” in 1991, proved genetically modified herpes simplex virus produced therapeutic benefits in treatment of malignant glioma. Since then, the development of oncolytic viruses to treat cancer has been attracting extensive attention. The basic concept of developing an oncolytic virus for treating cancer is to genetically modify a mildly pathogenic virus to achieve the tumor selectivity. Oncolytic viruses inhibit tumor growth by two mechanisms: oncolysis and anti-tumor immunity. Once an oncolytic virus enters a tumor, the virus replicates resulting in tumor cell death and lysis (oncolysis). And the cellular debris of the lyzed cells induces tumor-specific immunity, thus helping kill tumor cells in situ or attacking already metastasized tumor cells. Because of the unique features associated with oncolytic viruses, developing oncolytic viruses for treatment of cancer has been demonstrated to be a promising treatment strategy.

Up to date, new castle disease virus (NDV), herpes simplex virus (HSV), reovirus, vaccinia virus and adenovirus have been utilized to develop oncolytic viruses. Pre-clinical studies have demonstrated oncolytic viruses possess an excellent safety profile and are effective in treating various tumors in animals. However, the clinical performance of oncolytic viruses is generally poor. The caveats associated with currently available oncolytic viruses are attributed to the design strategies exploited for generation of oncolytic viruses. Currently, available oncolytic viruses were mainly developed by deleting one or more viral non-essential genes to confer the tumor selectivity to the virus. Viral non-essential genes are not required for the virus to grow in tissue culture. But the gene products of viral non-essential genes have been shown to directly or

Indirectly contribute to viral replication in vivo. Deleting non-essential genes from the viral genome allows the virus to selectively replicate in tumor cells, but the selectivity is achieved at the expenses of replication ability. That is why currently available oncolytic viruses generally perform poorly in clinic. In order to enhance the efficacy and broaden the application of oncolytic viruses in treatment of tumors, it is essential to maintain the integrity of the viral genome while not disrupting the temporal coordination of the viral gene expression. One option to achieve the goal is to confer tumor selectivity to the virus by adding exogenous sequences to regulate the expression of viral gene(s) such that the virus can only replicate in tumor cells.

Based on that concept, this disclosure is hereby provided

### SUMMARY OF THE INVENTION

One aspect of the present disclosure provides an oncolytic virus, which selectively replicates in and kill tumor cells effectively while the virus is safe to non-tumor cells.

Another aspect of the present disclosure provides nucleic acid fragments for preparing the above-mentioned oncolytic virus.

Another aspect of the present disclosure provides an oncolytic virus containing the aforementioned nucleic acid fragments.

Another aspect of the present disclosure provides a method for preparing the above-mentioned oncolytic virus.

Another aspect of the present disclosure provides the use of the above-mentioned oncolytic virus in tumor treatment.

Another aspect of the present disclosure provides a drug for treating cancer.

In the first aspect, the present disclosure provides an oncolytic virus, in which a first regulatory element is inserted into the viral genome. The first regulatory element comprises a tumor-specific promoter and a first nucleic acid sequence, which is driven by the tumor-specific promoter to express specific protease in target cancer cells.

It should be noted that insertion of the first regulatory element is located between two genes of the oncolytic virus. It can be inserted between two essential genes, or between one essential gene and one non-essential gene.

A second regulatory element is also inserted into the genome of the oncolytic virus. The second regulatory element comprises: a second nucleic acid sequence for encoding an extracellular secretion signal peptide and a third nucleic acid sequence for encoding a specific cleavage site. The specific cleavage site is recognized and cleaved by the specific protease.

The second regulatory element is in frame inserted between the first and second codons of one essential gene of the oncolytic virus. Therefore, a fusion protein will be produced once the regulated essential gene is expressed.

When the oncolytic virus enters non-tumor cells such as normal cells, the specific protease thereof is not expressed. Therefore, the extracellular secretion signal peptide within the fusion protein will direct the fusion protein to secrete to the outside of the cells once the fusion protein is produced, resulting in a trace amount of or no gene product of the regulated viral gene remaining in the cells. As a result, there is no viral replication or the virus replicates extremely poor in the cells.

When the oncolytic virus enters tumor cells, the specific protease will be robustly expressed, and the fusion protein once produced will be recognized and cleaved by the specific protease, thus allowing the regulated viral protein to remain within the cells and play its function to support viral replication.

The recombinant oncolytic virus provided in the present disclosure does not delete any viral gene from the viral genome. Therefore, it can replicate in tumor cells with a similar efficacy to that observed for the wild-type virus, thus killing tumor cells effectively.

The genome structure of the oncolytic virus provided in the present disclosure is shown in FIG. 1A.

In one or more embodiments of the present disclosure, the specific protease is selected from the group consisting of human rhinovirus 3C protease (HRV 3C protease), thrombin, factor Xa protease, tobacco etch virus protease (TEV protease) or recombinant PreScission protease.

Of course, it should be noted that the specific proteases related to the present disclosure is not limited to those described above. In some other embodiments, those skilled in the art can select suitable proteases as needed to be used in the present disclosure, which all fall into the protection scope of this disclosure.

Correspondingly, those skilled in the art can select suitable specific cleavage sites that can be recognized and cleaved by specific proteases as needed to apply to the present disclosure. For example,

When the specific protease is human rhinovirus 3C protease, the amino acid sequence of the specific cleavage site is: LEVLFQGP.

When the specific protease is thrombin, the amino acid sequence of the specific cleavage site is: LVPRGS.

When the specific protease is factor Xa protease, the amino acid sequence of the specific cleavage site is: IE/DGR (IEDGR or IDGR).

When the specific protease is tobacco etch virus protease, the sequence of the specific cleavage site is: ENLYFQG.

When the specific protease is recombinant PreScission protease, the sequence of the specific cleavage site is: LEVLFQGP.

In one or more embodiments of the present disclosure, the extracellular secretion signal peptide is selected from the group including interferon α2, interleukin 2, human serum albumin, human immunoglobulin heavy chain and luciferase extracellular secretion signal peptides.

Of course, it should be noted that the extracellular secretion signal peptides, which can be applied to the present disclosure, are not limited to those described above. In some other embodiments, those skilled in the art can select other appropriate extracellular secretion signal peptides as needed to apply to the present disclosure. As long as the extracellular secretion signal peptide can drive the protein fused with it to secrete to the outside of the cells, it should be within the protection scope of the present disclosure.

In one or more embodiments of the present disclosure, the first regulatory element further comprises an enhancer; the enhancer is inserted between the tumor-specific promoter and the specific protease encoding sequence. The enhancer is used to enhance the expression of the specific protease in tumor cells.

The present disclosure does not limit the enhancer types. Any enhancer that can enhance the expression of the downstream gene can be used in the present disclosure. In one or more embodiments, the enhancer is either CMV enhancer or SV40 enhancer. Of course, in other embodiments of the present disclosure, those skilled in the art can select other suitable enhancer as required, no matter which enhancer is selected, as long as its purpose is to be used for enhancing the expression of the specific protease in tumor cells, especially in tumor cells with low tumor-specific promoter activity, it falls within the scope of protection of the present disclosure.

In one or more embodiments of the present disclosure, the target tumor cells are lung cancer, liver cancer, breast cancer, gastric cancer, prostate cancer, brain tumor, human colon cancer, cervical cancer, and kidney cancer, ovarian cancer, head and neck cancer, melanoma, pancreatic cancer and esophageal cancer cells.

In one or more embodiments of the present disclosure, the tumor-specific promoter is selected from the group consisting of telomerase reverse transcriptase (hTERT), human epidermal growth factor receptor-2 (HER-2), E2F1, osteocalcin, carcinoembryonic antigen, survivin and ceruloplasmin promoters.

Of course, the tumor-specific promoters applied to the present disclosure are not limited to those mentioned above. In some other embodiments, those skilled in the art can select a suitable promoter that can specifically drive the expression of downstream genes in tumor cells as required. No matter which tumor-specific promoter is selected, it belongs to the protection scope of the present disclosure.

In one or more embodiments of the present disclosure, the oncolytic virus is selected from the group consisting of herpes simplex virus (such as herpes simplex virus type 1), adenovirus, vaccinia virus, newcastle disease virus, poliovirus, coxsackie virus, measles virus, mumps virus, vesicular stomatitis virus, and influenza virus.

It should be noted that virus types applied to the present disclosure are not limited to those mentioned above. In other embodiments, those skilled in the art can select a suitable oncolytic virus based on needed. But as long as it has the ability to infect and selectively tumor cells, no matter which virus is selected, it belongs to the protection scope of the present disclosure.

In addition, each virus contains several essential genes, and those skilled in the art can select genes other than the gene mentioned in the embodiments to make new oncolytic viruses by using the concepts provided in the present disclosure.

For example, when the oncolytic virus is herpes simplex virus, the essential gene is selected from the group consisting of envelope glycoprotein L, uracil DNA glycosylase, capsid protein, helicase proenzyme subunit, DNA replication initiation binding unwindase, derived protein of myristic acid, deoxyribonuclease, coat serine/threonine protein kinase, DNA packaging terminase subunit 1, coat protein UL16, DNA packaging protein UL17, capsid triplex subunit 2, major capsid protein, envelope protein UL20, nucleoprotein UL24, DNA packaging protein UL25, capsid mature protease, capsid protein, envelope glycoprotein B, single-stranded DNA-binding protein, DNA polymerase catalytic subunit, nuclear egress layer protein, DNA packaging protein UL32, DNA packaging protein UL33, nuclear egress membrane protein, large capsid protein, capsid triplex subunit 1, ribonucleotide reductase subunit 1, ribonucleotide reductase subunit 2, capsule host shutoff protein, DNA polymerase processing subunit, membrane protein UL45, coat protein VP13/14, trans-activating protein VP16, coatprotein VP22, envelope glycoprotein N, coat protein UL51, unwindase-primase primase subunit, envelope glycoprotein K, ICP27, nucleoproteinUL55, nucleoproteinUL56, transcription regulatory factorICP4, regulatory protein ICP22, envelope glycoprotein D, and membrane protein US8A.

Or, when the oncolytic virus is adenovirus, the essential gene is selected from the group consisting of early protein 1A, early protein 1B 19K, early protein 1B 55K, encapsidation protein Iva2, DNA polymerase, terminal protein precursor pTP, encapsidation protein 52K, capsid protein precursor pIIIa, pentomer matrix, core protein pVII, core protein precursor pX, core protein precursor pVI, hexonmer, proteinase, single-stranded DNA-binding protein, hexamer assembly protein 100K, protein 33K, encapsidation protein 22K, capsid protein precursor, protein U, fibrin, open reading frame 6/7 of regulatory protein E4, regulatory protein E4 34K, open reading frame 4 of regulatory protein E4, open reading frame 3 of regulatory protein E4, open reading frame 2 of regulatory protein E4, and open reading frame 1 of regulatory protein E4.

Or, when the oncolytic virus is vaccinia virus, the essential gene is selected from the group consisting of nucleotide reductase small-subunit, serine/threonine kinase, DNA-binding viral core protein, polymerase large-subunit, RNA polymerase subunit, DNA polymerase, sulfhydryl oxidase, hypothetical DNA-binding viral nucleoprotein, DNA-binding phosphoprotein, nucleoid cysteine proteinase, RNA helicase NPH-II, hypothetical metalloproteinase, transcription elongation factor, glutathione-type protein, RNA polymerase, hypothetical viral nucleoprotein, late transcription factor VLTF-1, DNA-binding viral nucleoprotein, viral capsid protein, polymerase small-subunit, RNA polymerase subunit rpo22 depending on DNA, RNA polymerase subunit rpo147 depending on DNA, serine/threonine protein phosphatase, IMV heparin-binding surface protein, DNA-dependent RNA polymerase, late transcription factor VLTF-4, DNA topoisomerase type I, mRNA capping enzyme large-subunit, viral core protein 107, viral core protein 108, uracil-DNA glycosylase, triphosphatase, 70 kDa small subunit of early gene transcription factor VETF, RNA polymerase subunit rpo18 depending on DNA, nucleoside triphosphate hydrolase-I, mRNA capping enzyme small-subunit, rifampicin target site, late transcription factor VLTF-2, late transcription factor VLTF-3, disulfide bond forming pathway, precursor p4b of core protein 4b, core protein 39 kDa, RNA polymerase subunit rpo19 depending on DNA, 82 kDa large subunit of early gene transcription factor VETF, 32 kDa small subunit of transcription factor VITF-3, IMV membrane protein 128, precursor P4a of core protein 4a, IMV membrane protein 131, phosphorylated IMV membrane protein, IMV membrane protein A17L, DNA unwindase, viral DNA polymerase processing factor, IMV membrane protein A21L, palmitoyl protein, 45 kDa large subunit of intermediate gene transcription factor VITF-3, RNA polymerase subunit rpo132 depending on DNA, RNA polymerase rpo35 depending on DNA, IMV protein A30L, hypothetical ATP enzyme, serine/threonine kinase, EEV mature protein, palm itoylated EEV membrane glycoprotein, IMV surface protein A27L, EEV membrane phosphate glycoprotein, IEV and EEV membrane glycoproteins, EEV membrane glycoprotein, disulfide bond forming pathway protein, hypothetical viral nucleoprotein, IMV membrane protein I2L, poxvirus myristoyl protein, IMV membrane protein L1R, late 16 kDa hypothetical membrane protein, hypothetical virus membrane protein H2R, IMV membrane protein A21L, chemokine-binding protein, epidermal growth factor-like protein, and IL-18 binding protein.

Or, when the oncolytic virus is coxsackie virus, the essential gene is selected from the group consisting of protein Vpg, core protein 2A, protein 2B, RNA unwindase 2C, protein 3A, proteinase 3C, reverse transcriptase 3D, coat protein Vp4, and protein Vp1.

Or, when the oncolytic virus is measles virus, the essential gene is selected from the group consisting of nucleoprotein N, phosphoprotein P, matrix protein M, transmembrane glycoprotein F, transmembrane glycoprotein H, and RNA-dependent RNA polymerase L.

Or, when the oncolytic virus is mumps virus, the essential gene is selected from the group consisting of nucleoprotein N, phosphoprotein P, fusion protein F, and RNA polymerase L.

Or, when the oncolytic virus is vesicular stomatitis virus, the essential gene is selected from the group consisting of glycoprotein G, nucleoprotein N, phosphoprotein P and RNA polymerase L.

Or, when the oncolytic virus is poliovirus, the essential gene is selected from the group consisting of capsid protein VP1, capsid protein VP2, capsid protein VP3, cysteine protease 2A, protein 2B, protein 2C, protein 3A, protein 3B, proteinase 3C, protein 3D, and RNA-directed RNA polymerase.

Or, when the oncolytic virus is influenza virus, the essential gene is selected from the group consisting of hemagglutinin, neuraminidase, nucleoprotein, membrane protein M1, membrane protein M2, polymerase PA, polymerase PB1-F2, and polymerase PB2.

In one or more embodiments of the present disclosure, a second regulatory element is inserted between the first and second codons of the open reading frame of one or more essential genes of the oncolytic virus.

In some embodiments of the present disclosure, the regulated viral essential genes can be more than one, and when several viral genes are regulated, the second regulatory element should be accordingly inserted between the first and second codons of the open reading frame of each essential gene.

In one or more embodiments of the present disclosure, the oncolytic virus is herpes simplex virus type 1, the essential gene is ICP27, the tumor-specific promoter is telomerase reverse transcriptase promoter, and the specific protease is the human rhinovirus 3C protease, the extracellular secretion signal peptide is interferon α2 signal peptide, the amino acid sequence of the specific cleavage site is LEVLFQGP; the second regulatory element is located between the first and second codons of the open reading frame of the essential gene ICP27. The first regulatory element is located downstream the essential gene.

In one or more embodiments of the present disclosure,

the nucleotide sequence of the telomerase reverse transcriptase promoter is shown in SEQ ID NO: 4.

The amino acid sequence of human rhinovirus 3C protease is shown in SEQ ID NO: 5.

The nucleotide sequence of the open reading frame of human rhinovirus 3C protease is shown in SEQ ID NO: 6.

The amino acid sequence of the interferon α2 signal peptide is shown in SEQ ID NO: 7; and the nucleotide sequence of the second nucleic acid sequence is shown in SEQ ID NO: 8.

The nucleotide sequence of the third nucleic acid sequence is shown in SEQ ID NO: 9.

In other aspect, the present disclosure provides a nucleic acid fragment for preparing the oncolytic virus, wherein the nucleic acid fragment consists of the 5′ UTR of the essential gene, ATG, the second regulatory element, the remaining portion of the open reading frame of the essential gene without ATG, an exogenous Poly (A), the first regulatory element followed by the 3′ UTR of the regulated essential gene. The 5′ and 3′ UTR sequences are used to facilitate the homologous recombination between the nucleic acid fragment-containing plasm id and viral genome for generation of the oncolytic virus.

In another aspect, the present disclosure provides an oncolytic virus containing the above-mentioned nucleic acid fragment.

In another aspect, the present disclosure provides a method for preparing oncolytic virus as described above, which comprises: infection of complementing cells with parental virus followed by transfection of the cells with plasmid DNA containing the nucleic acid fragment, and screening, confirmation and propagation of the oncolytic virus.

Herein, the genome of the parent virus lacks the regulated viral essential genes as compared to the genome of the wild-type virus.

Complementing cells constitutively expresses the regulated viral essential gene.

In another aspect, the present disclosure provides the use of the oncolytic virus as described above as a drug for killing tumor cells in vitro.

In another aspect, the present disclosure provides a drug for treating tumors, comprising the oncolytic virus as described above and pharmaceutically acceptable excipients.

Those skilled in the art can select an appropriate pharmaceutically acceptable excipient as required, and it is not limited in the present disclosure.

In one or more embodiments of the present disclosure, the drug also includes gene therapies or vaccines.

In another aspect, the present disclosure provides a method of treating a disease in animals including:

Administration of the drugs provided in the present disclosure to animals,

wherein the disease is lung cancer, gastric cancer, liver cancer, rectal cancer, breast cancer, prostate cancer, brain tumor, colon cancer, cervical cancer, kidney cancer, ovarian cancer, head and neck cancer, melanoma, pancreatic cancer or esophageal cancer.

### BRIEF DESCRIPTION OF DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which

FIG. 1: Schematic representation of the genome structure of the recombinant oncolytic virus provided in the present disclosure. A: generalized genome structure of oncolytic viruses provided in this disclosure, wherein the first regulatory element was located downstream the regulated essential gene, and the second regulatory element is located between the first and second codons of the open reading frame of the regulated essential gene. B: a specific embodiment of A, wherein the virus was HSV-1, the tumor-specific promoter was hTERT promoter, the enhancer was CMV enhancer, and the specific protease was HRV-3C protease, the regulated essential gene was ICP27, the extracellular secretion signal peptide was interferon α2 signal peptide; the specific cleavage site was specifically recognized and cleaved by HRV-3C protease. The oncolytic virus constructed was named as oHSV-BJS.

FIG. 2: Schematic showing of the parental plasmid pcDNA3.1-EGFP unitized for constructing a plasmid expressing HSV-1 ICP27. In the plasmid, EGFP is constitutively expressed under the control of CMV promoter and the plasmid contains the neomycin-resistant gene expression sequence

FIG. 3: ICP27 expression from oncolytic virus oHSV-BJS in African green monkey kidney cells (Vero, normal cells). Vero cells were infected with 3 MOI (virus/cell) HSV-1 wild-type virus KOS or oncolytic virus oHSV-BJS. One day later, the cells were collected, RNAs and proteins were isolated. ICP27 mRNA was detected by reverse transcription combined with semi-quantitative PCR (A in the FIG.), and ICP27 protein detected by Western blotting (B in the FIG.).

FIG. 4: Expression of HRV-3C in four tumor cells. Four tumor cells were infected with 3 MOI oncolytic virus oHSV-BJS or KOS. 24 hours after infection, total RNA and protein were isolated. HRV-3C mRNA was analyzed by semi-quantitative PCR, and t ICP27 protein detected by Western blotting. A: HRV-3C mRNA; B: ICP27 protein.

FIG. 5: Replication kinetics of wild-type virus KOS and oncolytic virus oHSV-BJS in tumor cells. Four tumor cells were infected with 0.1 MOI KOS or oHSV-BJS, respectively. At different day after infection, the cells and culture medium were collected. The virus titer for each viral stock was determined. A: cervical tumor Hela cells; B: cervical squamous carcinoma siHa cells; C: breast cancer SK-BR3 cells; D: breast cancer ME-180 cells.

FIG. 6: Inhibition of tumor growth by oncolytic virus oHSV-BJS in animal tumor models. Tumor animal models were established. After the tumor grew to 50-80 mm³, the oncolytic virus was injected into the tumor every 3 days for a total of 3 times. PBS (without oncolytic virus) was injected as a negative control. After the oncolytic virus was injected, the tumor size was tested twice a week. When the negative control animals needed to be euthanized, the experiment ended. A tumor growth curve was plotted based on the tumor size (A: lung cancer; B: gastric cancer; C: liver cancer; D: rectal cancer), and the relative inhibition rate (E) was calculated by comparing the tumor size in the test group at the end of the test with that observed in the negative control.

### DETAILED DESCRIPTION OF THE EMBODIMENTS

In order to demonstrate the features of the present disclosure, its nature and various advantages, exemplary embodiments were executed and are described in details below. All experiments were conducted using standard methods as described in literature. Reagents were purchased from commercial providers and used according to the instructions of the manufacturer.

As used herein, terms “base sequence” and “nucleotide sequence” can be used interchangeably, and generally refer to the composition and order of nucleotides arranged in DNA or RNA.

The term “primer” refers to a synthetic oligonucleotide, which is required for de novo nucleic acid synthesis. After binding to a polynucleotide template, the primer is extended in 5′ to 3′ direction along the template catalyzed by DNA polymerase, hereby producing an extended duplex. Nucleotide addition during the extension is determined by the sequence of the template. A primer is typically 18-23 nucleotides in length. However, a primer length is determined by several factors including the nucleotide composition and the melting point of the primer, and the downstream application of the PCR product after amplified.

The term “promoter” generally refers to a DNA sequence that is located upstream the coding region of a gene, can be specifically identified and bound to by an RNA polymerase, and is required by transcription.

The term “enhancer” refers to a DNA sequence that increases transcription frequency of the gene interlocked therewith. The enhancer enhances the transcription by increasing the activity of a promoter. An enhancer may be located either at the 5′ or the 3′end of a gene, and even may exist as an intron within a gene. An enhancer might significantly affect gene expression, which might increase the gene transcription by 10-200 folds, or even by thousand times.

Terms “subject”, “individual”, and “patient” can be used interchangeably herein, and refer to a vertebrate, preferably a mammal, most preferably human. The mammal comprises, but is not limited to, mouse, ape, human, domesticated animal, or farm-raised livestock.

The features and its nature of the present disclosure are described in detail below with reference to examples.

### Example 1

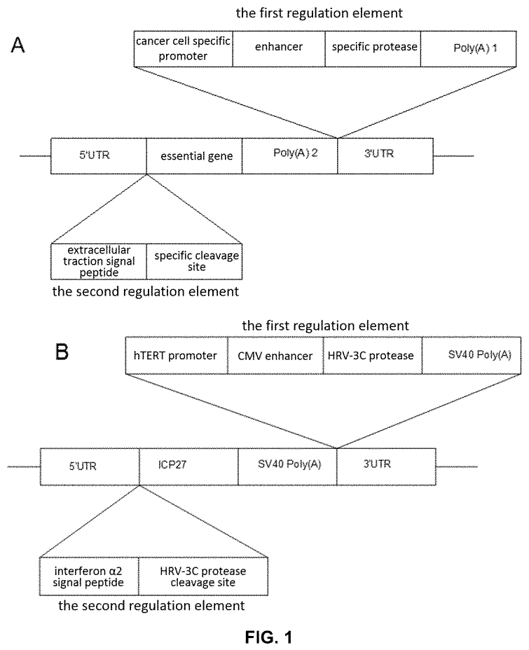

The oncolytic virus provided in this example was generated by genetically engineering wild-type herpes simplex virus type 1 KOS. The genome of the oncolytic virus oHSV-BJS contains the following elements refer to FIG. 1B).

(1) A first regulatory element is located downstream the essential gene ICP27 of the oncolytic virus; and the first regulatory element includes: tumor specific promoter, namely hTERT promoter, an enhancer, namely CMV enhancer, a nucleic acid sequence for encoding the specific protease, namely human rhinovirus 3C protease (HRV-3C protease) and BGH Poly(A).

Herein, the base sequence of hTERT promoter is shown in SEQ ID NO: 4;

the base sequence of the CMV enhancer is shown in SEQ ID NO: 10;

the nucleic acid fragment encoding HRV-3C protease is shown in SEQ ID NO: 6; and

the amino acid sequence of HRV-3C protease is shown in SEQ ID NO: 5.

(2) A second regulatory element is located between the first and second codons of the open reading frame of the essential gene ICP27 of the oncolytic virus; and the second regulatory element includes: the second nucleic acid sequence for encoding an extracellular secretion signal peptide, namely the interferon α2 signal peptide, and the third nucleic acid sequence for encoding the specific cleavage site sequence.

Herein, the amino acid sequence of the interferon α2 signal peptide is shown in SEQ ID NO: 7;

the nucleotide sequence of the second nucleic acid sequence for encoding the interferon α2 signal peptide is shown in SEQ ID NO: 8;

the amino acid sequence of the specific cleavage site is LEVLFQGP; and

the nucleotide sequence of the third nucleic acid sequence for encoding the specific cleavage site, is TTAGAAGTTCTTTTTCAAGGTCCT.

When the oncolytic virus infects normal cells, the HRV-3C protease is not expressed. Therefore, the specific cleavage site will be not cleaved, and the interferon α2 extracellular secretion signal peptide will direct the secretion of the ICP27 fusion protein to the outside of the cells, resulting in no viral replication. Therefore, the virus is safe to normal cells. When the oncolytic virus infects tumor cells, the HRV-3C protease is specifically expressed under the control of hTERT promoter, and the specific cleavage site will be recognized and cleaved by the expressed HRV-3C protease, and the ICP27 protein can be partitioned and localized normally in the tumor cells. Therefore, the oncolytic virus replicates normally and kill target tumor cells.

The preparation of the above-mentioned oncolytic virus oHSV-BJS provided in this example was as follows.

(1) Preparation of Complementing Cells Expressing HSV-1 ICP27

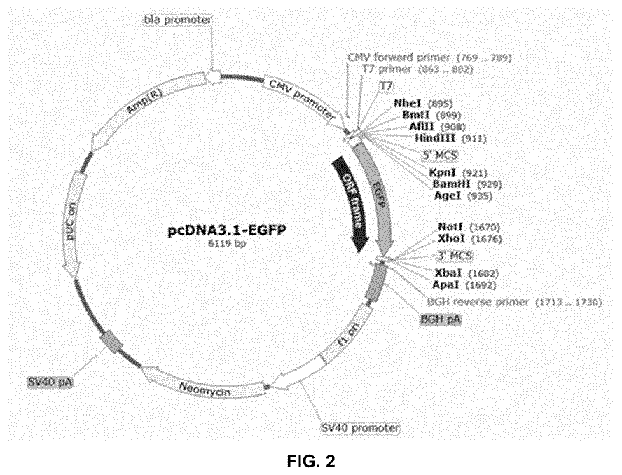

(a) plasmid construction: Using the DNA of wild-type herpes simplex virus type 1 KOS as a template, the encoding region of ICP27 was amplified by PCR, and inserted into HindIII and XbaI sites of the plasm id pcDNA3.1-EGFP (FIG. 2) to replace EGFP. The recombinant plasmid was named as ICP27 expression plasmid. The expression of ICP27 from the plasm id is driven under the control of CMV promoter.

(b) G418 dose determination for selection: Vero cells were treated with G418 of different concentrations, the culture medium containing G418 was replaced every three days with media containing G418 of different concentrations, and cell death was monitored every day. The minimal concentration of G418 required for all the cells to die after 6 days of G418 treatment was determined. Such a concentration of G418 (500 μg/ml) was utilized for complementing cell establishment.

(c) Cell line establishment: 3.5×10⁵ Vero cells were seeded into each well of a 6-well cell culture plate and cultured overnight in an antibiotic-free culture medium, and 4 μg of the ICP27 expression plasmid DNA obtained from step (a) were transfected into cells in each well using Lipofectamine 2000. After 24 hours of culture, cells in each well were harvested, and diluted by 20, 40, or 60-fold. Cells were cultured in the culture medium containing 500 μg/ml G418, and the medium replaced with fresh medium containing G418 every 3 days. After 6-7 times of medium change, the clones were collected and propagated step by step from the 24-well plate to T150 tissue culture flasks. Subsequently, the protein was isolated, and the expression of ICP27 detected by western blotting. The Cells with the highest level of ICP27 expression were selected as the complementing cells to support the growth and replication of replication-defective viruses in which ICP27 are not expressed; the cells were named as C_{ICP27}

The complementing C_{ICP27} cells have been preserved at China Center for Type Culture Collection (CCTCC), Wuhan University, Luojiashan, Wuchang, Wuhan City on Apr. 24, 2019 with a preservation number of CCTCC NO. C201974.

(2) Preparation of the Parental Virus HSV-EGFP

The wild-type type 1 herpes simplex virus KOS was used as the starting material. The recombinant parental virus HSV-EGFP was obtained by homologous recombination between plasmid and KOS genome. In HSV-EGFP, HSV-ICP27 was replaced by EGFP. HSV-EGFP served as the parental virus for generating the oncolytic viruses provided in this disclosure. The detailed manipulations were as the follows.

(a) the first nucleic acid fragment was synthesized and its nucleotide sequence is shown in SEQ ID NO. 1:

The fragment includes the following elements: ICP27 5′ sequence, CMV promoter, Kozak sequence, EGFP encoding frame, BGH Poly(A) and ICP27 3′ sequence.

Sequence seen in SEQ ID NO. 1:

site 1-6: irrelevant sequence, increasing the end length to facilitate enzyme digestion;

site 7-12: Xho1 site, C/TCGAG;

site 13-575: ICP27 5′ UTR without ICP27 endogenous promoter;

site 576-1163: CMV promoter;

site 1164-1174: interval sequence;

site 1175-1180: Kozak sequence, increasing protein expression;

site 1181-1900: EGFP encoding frame;

site 1901-2145: BGH Poly(A);

site 2146-2667: ICP27 3′ UTR;

site 2668-2673: HindIII site, A/AGCTT; and

site 2674-2679: irrelevant sequence, increasing the end length to facilitate enzyme digestion.

(b) the first fragment was cleaved and ligated to the HindIII and Xho1 sites of the pcDNA3.1-EGFP plasmid, and the resulting recombinant plasmid was named as EGFP expression plasmid.

(c) 3.5×10⁵ complementing C_{ICP27} cells were seeded into each well of 6-well cell culture plate and cultured overnight in an antibiotic-free culture medium.

(d) the cells were infected with 0.1, 0.5, 1, 3 MOI wild-type virus KOS (virus/cell), respectively. One hour later, the above-mentioned EGFP expression plasmid (4 μg DNA/well) was transfected into the cells using Lipofectamine 2000. After 4 hours of incubation, the transfection mixture was replaced with complete medium. When all the cells became spherical, the cells and culture medium were collected. The mixture was centrifuged after three cycles of freeze-thawing and the supernatant collected, The virus stocks were diluted, and infected complementing C_{ICP27} cells. Viruses were separated using plaque separation method. 4-5 days later, the virus plaque with the strongest green fluorescence was selected and picked under a fluorescence microscope. The obtained virus plaque was subjected to 2 or 3 rounds of screening to obtain pure virus plaques. The virus was propagated. The recombinant virus with ICP27 replaced by EGFP was named as HSV-EGFP. HSV-EGFP served as the parental virus for generation of oncolytic virus oHSV-BJS provided in this disclosure.

(3) Construction of Recombinant Plasm Id Containing the First Expression Regulatory Element and the Second Regulatory Element

(a) the TA cloning plasmid was modified such that the multiple cloning site only contains the XhoI site, and the resulting plasmid named as TA-XhoI plasmid.

(b) the second nucleic acid fragment was synthesized with sequence shown in SEQ ID NO:2. The fragment includes the following elements:

ICP27 5′ UTR including the endogenous promoter, ATG, the second nucleic acid sequence for encoding interferon α2 extracellular secretion signal peptide, the third nucleic acid sequence for encoding the specific cleavage site recognized and cleaved by HRV-3C protease, ICP27 open reading frame sequence without ATP, SV40 Poly(A), ICP27 3′ UTR, wherein there was a HindIII site between SV40 Poly(A) and ICP27 3′ UTR, which is used to insert the first regulatory element. Detailed information of each element in the fragment is as follows:

site 1-6: Xho1 site;

site 7-677: ICP27 5′ UTR containing endogenous ICP27 promoter;

site 678-746: the second nucleic acid sequence for encoding the interferon α2 signal peptide;

site 747-770: the third nucleic acid sequence for encoding the specific cleavage site of HRV-3C protease;

site 771-2306: ICP27 open reading frame (without start codon ATG);

site 2307-2753: SV40 Poly(A);

site 2754-2759: HindIII site;

site 2760-3279: ICP27 3′ UTR; and

site 3280-3285: Xho1 site.

The second nucleic acid fragment was cleaved by Xho 1, ligated into the Xho1 site of plasmid TA-XhoI, and the resulting plasmid was named as pTA-XhoI-S-ICP27.

(c) the third nucleic acid fragment was synthesized with sequence shown in SEQ ID NO:3. It contains the following elements: hTERT promoter, CMV enhancer, Kozak sequence, the nucleic acid sequence for encoding the HRV-3C protease and SV40 Poly(A). Detailed information of each element in the fragment is as follows:

site 1-6: HindIII site;

site 7-432: hTERT promoter;

site 433-527: CMV enhancer;

site 528-539: Kozak sequence;

site 540-1088: nucleic acid sequence for encoding HRV-3C protease;

site 1089-1544: SV40 Poly(A); and

site 1545-1550: HindIII site.

The third nucleic acid fragment was cleaved by HindIII and ligated into pTA-XhoI-S-ICP27, and the obtained plasmid was named as pTA-XhoI-S-ICP27-3C plasmid.

(4) Construction of Recombinant Oncolytic Herpes Viruses

(a) 3.5×10⁵ complementing C_{ICP27} cells were seeded into each well of a 6-well cell culture plate and cultured overnight in an antibiotic-free culture medium.

(b) the complementing C_{ICP27} cells were infected with 0.1, 0.5, 1, 3 MOI (virus/cell) of the parent virus HSV-EGFP, respectively. After 1 hour incubation, pTA-XhoI-S-ICP27-3C DNA (4 μg DNA/well) was transfected into the cells using Lipofectamine 2000. After 4 hours of incubation, the transfection mixture was replaced with complete medium. When all the cells became spherical, the cells and culture medium were collected. The mixture was centrifuged after three cycles of freeze-thawing, the supernatant collected. Virus stocks were diluted, and infected the complementing C_{ICP27} cells. The viruses were isolated using plaque separation method. 4-5 days after infection, virus plaques without green fluorescence under a fluorescence microscope were picked. The obtained virus plaques were subjected to 2 or 3 rounds of screening to obtain pure virus plaques, the virus was propagated and expanded, the infected cell DNA was isolated, and the recombinant oncolytic viruses were confirmed by PCR amplification and sequencing, and the oncolytic virus was named as oHSV-BJS.

The recombinant oncolytic virus oHSV-BJS was preserved in the Chinese Center of Type Culture Collection (CCTCC), a Wuhan University, Luojiashan, Wuchang, Wuhan, China on Apr. 24, 2019. The preservation number is CCTCC NO: V201920.

### Experimental Example 1

Detection of the expression of ICP27 from the recombinant oncolytic virus oHSV-BJS in Vero cells

Method: Vero was infected with 3 MOI wild-type KOS and oncolytic virus oHSV-BJS, respectively. One day after infection, cells were collected, RNAs and proteins were isolated. ICP27 mRNA was detected by reverse transcription combined with semi-quantitative PCR, and tICP27 protein detected by Western blotting. For mRNA and protein detection, β-actin was used as the loading control.

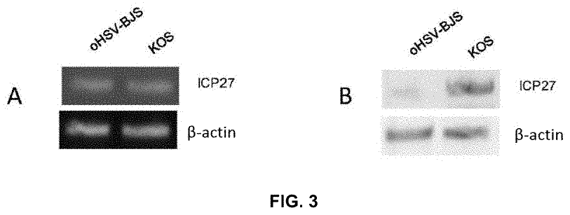

Results: oHSV-BJS there was no significant difference in ICP27 mRNA level in oHSV-BJS-infected vero cells compared to KOS-infected vero cells (FIG. 3A). However, ICP27 protein in Vero cells infected with oHSV-BJS was significantly lower than that observed in Vero cells infected with KOS (FIG. 3B)). The results suggest ICP27 protein once produced is secreted to the outside of the cells.

### Experimental Example 2

Detection of the Expression of HRV-3C Protease and ICP27 in Tumor Cells

Method: Four tumor cells were infected with 3 MOI oncolytic virus oHSV-BJS or KOS. 24 hours after infection, total RNA and protein were isolated. HRV-3C mRNA was analyzed by semi-quantitative PCR, and t ICP27 protein detected by Western blotting. For mRNA and protein detection, β-actin was used as the loading control.

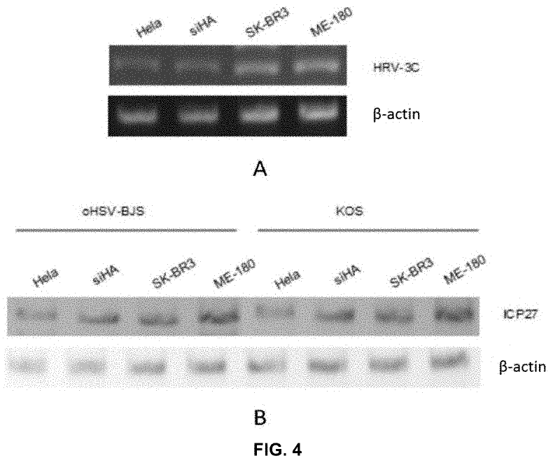

Results: HRV-3C mRNA was expressed to a detectable level in all the four tumor cells infected with oHSV-BJS (FIG. 4A). ICP27 protein was accumulated to a detectable level in all the four tumor cells infected with the oncolytic virus oHSV-BJS. Moreover, there was no significant difference in ICP27 protein between oncolytic virus oHSV-BJS and KOS infected cells for a given cell type (FIG. 4B).

The results demonstrate that HRV-3C was specifically expressed in tumor cells and cleaved ICP27 fusion protein.

### Experimental Example 3

Replication Kinetics of Recombinant Oncolytic Virus oHSV-BJS

Method: tumor cells were infected with 0.1 MOI KOS or oHSV-BJS, respectively. At different day after infection, the cells and culture medium were collected, and the viruses remaining in the cells were released into the culture medium through three cycles of −80/37° C. freeze-thawing. The complementing C_{ICP27} cells were then infected with the virus, and the virus titer (plaque forming unit/ml, PFU/ml) was determined by plaque assay.

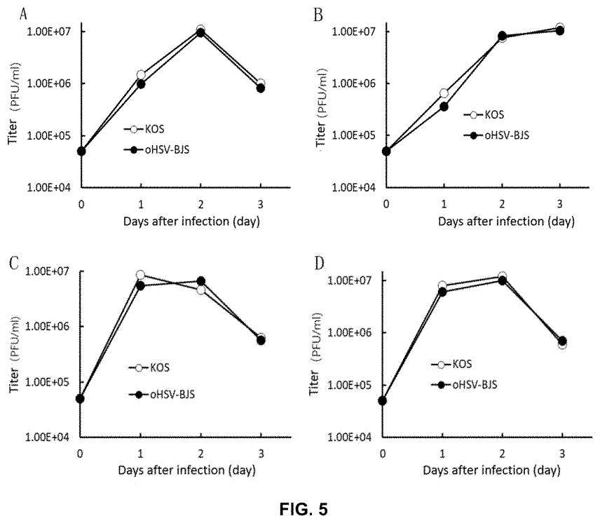

Results: with the replication kinetics of oHSV-BJS are quite different from one cell to another cell type. But for a given cell type, there is no significant difference in viral replication in bother oHSV-BJS-infected and KOS-infected cells (FIG. 5A-D). the results indicate that genetic modification used for generating the oncolytic virus provided in this disclosure does not significantly alter the replication capacity in tumor cells.

### Experimental Example 4

The Ability of Recombinant Oncolytic Virus oHSV-BJS to Kill Tumor Cells

Methods: tumor cells were infected by MOI 0.25 or 0.5 KOS or oHSV-BJS respectively. Cell viability was assayed at different day after infection

0.25 or 0.5 MOI oHSV-BJS shows a varied ability to kill different tumor cells. But for a given cell type, the efficiency of oHSV-BJS in killing cells was basically the same as that observed for KOS (Table 1-4). the results indicate that oncolytic virus oHSV-BJS retains the ability of wild-type KOS virus to kill tumor cells.

TABLE 1
Viability of (%) of Hela cells infected with oHSV-BJS
at different day after infection

| virus (MOI 0.25) | oHSV-BJS | KOS |
|---|---|---|
| the first day | 58 ± 3.0 | 46 ± 2.0 |
| the second day | 38 ± 2.0 | 29 ± 1.4 |
| the third day | 17 ± 0.8 | 11 ± 0.6 |
| the fourth day | 4 ± 0.3 | 0 |
| virus (MOI 0.5) | oHSV-BJS | KOS |
| the first day | 52 ± 2.7 | 39 ± 1.8 |
| the second day | 28 ± 1.8 | 22 ± 1.1 |
| the third day | 9 ± 0.1 | 5 ± 0.3 |
| the fourth day | 0 | 0 |

TABLE 2
Viability (%) of siHA cells infected with oHSV-BJS
at different day after infection

| virus (MOI 0.25) | oHSV-BJS | KOS |
|---|---|---|
| the first day | 22 ± 1.4 | 16 ± 1.0 |
| the second day | 6 ± 0.4 | 0 |
| the third day | 0 | 0 |
| virus (MOI 0.5) | oHSV-BJS | KOS |
| the first day | 8 ± 0.4 | 4 ± 0.3 |
| the second day | 1 ± 0.1 | 0 |
| the third day | 0 | 0 |

TABLE 3
Viability (%) of SK-BR3 cells infected with oHSV-BJS
at different day after infection

| virus (MOI 0.25) | oHSV-BJS | KOS |
|---|---|---|
| the first day | 21 ± 1.3 | 6 ± 0.4 |
| the second day | 0 | 0 |
| the third day | 0 | 0 |
| virus (MOI 0.5) | oHSV-BJS | KOS |
| the first day | 3 ± 0.1 | 5 ± 0.3 |
| the second day | 0 | 0 |
| the third day | 0 | 0 |

TABLE 4
viability (%) of ME-180 cells infected with oHSV-BJS
at different day after infection

| virus (MOI 0.25) | oHSV-BJS | KOS |
|---|---|---|
| the first day | 12 ± 0.8 | 10 ± 0.5 |
| the second day | 4 ± 0.3 | 0 |
| the third day | 0 | 0 |
| virus (MOI 0.5) | oHSV-BJS | KOS |
| the first day | 5 ± 0.5 | 5 ± 0.4 |
| the second day | 1 ± 0.10 | 0 |
| the third day | 0 | 0 |

### Experimental Example 5

Effect of Recombinant Oncolytic Virus oHSV-BJS on the Viability of Normal Cells

Methods: Vero cells or primary human corneal epidermal cells were infected with oncolytic virus oHSV-BJS (2 MOI) or wild virus KOS (0.5 MOI) respectively. Viability of the cells infected with oHSV-BJS were assayed 3 days after infection while the viability of the cells infected with wild virus KOS were measured 2 days after infection. All Vero cells or primary human corneal epidermal cells died 2 days after KOS infection. But the viability of the cells infected with the oncolytic virus oHSV-BJS was basically the same as that of observed for mock treatment (not treated) (Table 5). The results indicate that oncolytic virus oHSV-BJS is safe to normal cells.

TABLE 5
viability (%) of normal cells 3 days after oHSV-BJS
infection or 2 days after KOS infection

| virus | oHSV-BJS | KOS | untreated |
|---|---|---|---|
| Vero cells | 95 ± 2 | 0 | 98 ± 2 |
| corneal epidermal cells | 97 ± 2 | 0 | 97 ± 1 |

### Experimental Example 6

Detection of the Inhibition of Tumor Growth by Oncolytic Virus oHSV-BJS In Vivo

Method: To establish animal tumor models of lung cancer (A549 cells), gastric cancer (NCI-N87 cells), liver cancer (SK-HEP-1 cells) and rectal cancer (HCT-8), tumor cells were subcutaneously inoculated into mice. When the tumors grew to 40-120 mm³, oncolytic virus oHSV-BJS was injected into the tumors once every 3 days for a total of 3 times of injection, 1.2×10⁷ infection units (suspended in 40μl PBS) were injected for each time at multiple sites. Mice injected with PBS (without oncolytic virus) were used as negative control, with 8 animals in each experiment group. After the injection of oncolytic virus, the tumor sizes were measured twice a week (the relative tumor size was defined as 1 at the first injection) for a total of 17-32 days (depending on the time when the animal in the negative control group needed to be euthanized). The tumor growth curve was plotted according to the tumor size. At the end of the experiment, the tumor size was measured and compared with that in the negative control, and the inhibition rate was calculated.

inhibition rate (%)=(tumor volume of negative control group−tumor volume of test group)/tumor volume in negative control group×100%.

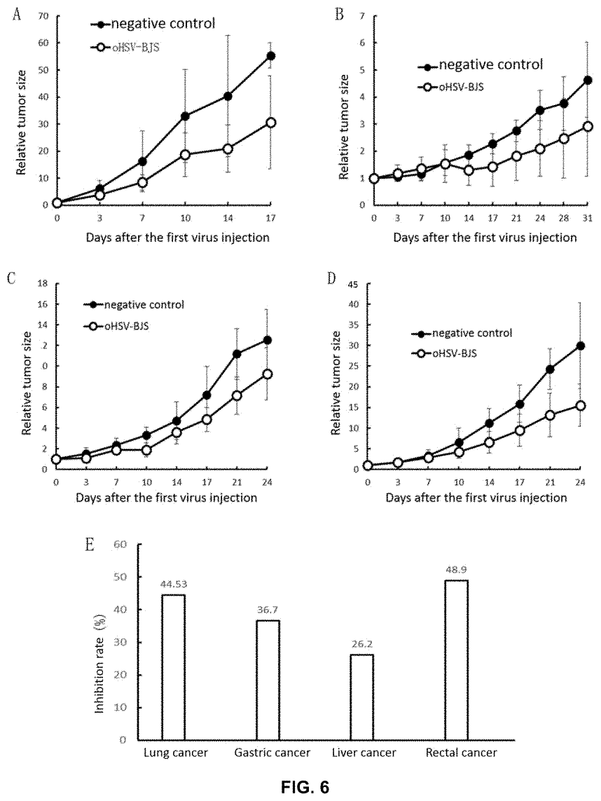

oHSV-BJS slowed down the growth of lung, gastric, liver and rectal tumors (FIG. 6A-D). The inhibition of oHSV-BJS on lung, gastric, liver and rectal tumor were 45%, 37%, 26% and 49%, respectively (FIG. 6E). The results showed that oHSV-BJS can inhibit the proliferation of various tumors.

The foregoing descriptions are only typical examples of the present disclosure, and are not intended to limit the scope of the present disclosure. Any modification, equivalent replacement, improvement, etc. made within the concept and principle of the present disclosure shall be included in the protection scope of the present disclosure.

### INDUSTRIAL APPLICABILITY

The oncolytic virus provided by the present disclosure can be produced on industrial scale. The oncolytic virus has not deleted any genes (either essential or non-essential genes) from its original genome. It can replicate with high capability and kill tumor cells, which can be used to treat cancer and it is safe to non-cancer cells.

1-21. (canceled)

22. An oncolytic virus, wherein a first regulatory element is inserted into the viral genome thereof, wherein the first regulatory element comprises a tumor-specific promoter and a first nucleic acid sequence. The first nucleic acid sequence is driven by the tumor-specific promoter to express the specific protease in target tumor cells;
a second regulatory element is further inserted into the viral genome, wherein the second regulatory element comprises a second nucleic acid sequence for encoding a specific cleavage site; and the specific cleavage site is recognized and cleaved by the specific protease; and
the second regulatory element is located between the first and second codons of the regulated essential gene of the oncolytic virus.

23. The oncolytic virus of claim 22, wherein the specific protease is selected from the group including human rhinovirus 3C protease, thrombin, factor Xa protease, tobacco etch virus protease or recombinant PreScission protease.

24. The oncolytic virus of claim 22, wherein the extracellular secretion signal peptide is either interferon α2, interleukin 2, human serum albumin, human immunoglobulin heavy chain extracellular secretion signal peptide, or luciferase extracellular secretion signal peptide derived from marine copepods.

25. The oncolytic virus of claim 22, wherein the second regulatory element is located between the first and second codons of the open reading frame of the regulated essential gene of the oncolytic virus.

26. The oncolytic virus of claim 22, wherein the first regulatory element further comprises an enhancer, and the enhancer is located between the tumor-specific promoter and the encoding sequence of the specific protease, the enhancer is used to enhance expression of the specific protease in target tumor cells; and
preferably, the enhancer is either CMV enhancer or SV40 enhancer.

27. The oncolytic virus of claim 22, wherein the target tumor cells are lung cancer, liver cancer, breast cancer, gastric cancer, prostate cancer, brain tumor, human colon cancer, cervical cancer, kidney cancer, ovarian cancer, head and neck cancer, melanoma, pancreatic cancer, or esophageal cancer cells.

28. The oncolytic virus of claim 22, wherein the tumor-specific promoter is selected from the group consisting of telomerase reverse transcriptase, human epidermal growth factor receptor-2, E2F1, osteocalcin, carcinoembryonic antigen, survivin and ceruloplasmin promoters.

29. The oncolytic virus of claim 22, wherein the oncolytic virus is selected from the group consisting of herpes simplex virus, adenovirus, vaccinia virus, new castle disease virus, poliovirus, coxsackie virus, measles virus, mumps virus, vesicular stomatitis virus and influenza virus.

30. The oncolytic virus of claim 22, wherein the second regulatory element is inserted between the first and second codons of one or more essential genes of the oncolytic virus.

31. The oncolytic virus of claim 22, wherein the oncolytic virus is herpes simplex virus type 1, the essential gene is ICP27, the tumor-specific promoter is telomerase reverse transcriptase promoter, the specific protease is human rhinovirus 3C protease, the extracellular secretion signal peptide is interferon α2 signal peptide, the amino acid sequence of the specific cleavage site is LEVLFQGP; the second regulatory element is located between the first and second codons of the open reading frame of the regulated essential gene; and the first regulatory element is located downstream the regulated essential gene.

32. The oncolytic virus of claim 31, wherein
the nucleotide sequence of the telomerase reverse transcriptase promoter is shown in SEQ ID NO: 4;
the amino acid sequence of the human rhinovirus 3C protease is shown in SEQ ID NO: 5;
the nucleotide sequence of the open reading frame of the human rhinovirus 3C protease is shown in SEQ ID NO: 6;
The amino acid sequence of interferon α2 extracellular secretion signal peptide is shown in SEQ ID NO: 7; the nucleotide sequence of the second nucleic acid sequence is shown in SEQ ID NO: 8; and
the nucleotide sequence of the third nucleic acid sequence is shown in SEQ ID NO: 9.

33. The oncolytic virus of claim 22, wherein the insertion of the first regulatory element is located between two genes of the oncolytic virus.

34. The oncolytic virus of claim 33, wherein the two genes can both be essential genes, or one is an essential gene and the other is a non-essential gene.

35. A nucleic acid fragment for preparing the oncolytic virus of claim 22, wherein the nucleic acid fragment consists of the 5′ UTR of the regulated essential gene, ATG, the second regulatory element, the remaining portion of the open reading frame of the regulated essential gene without ATG, an exogenous Poly(A) and the first regulatory element followed by the 3′ UTR of the regulated essential gene. The 5′ and 3′ UTRs provide the sequence basis for homologous recombination between plasmid DNA and the parental viral genome for generation of the oncolytic viruses provided in the disclosure.

36. A drug for treating tumors, wherein the drug comprises the oncolytic virus of claim 1 and pharmaceutically acceptable excipients.

37. The drug of claim 36, wherein the drug further comprises gene therapy drug or vaccine.


SEQUENCE LISTING (10 sequences)

SEQ ID NO: 1 - DNA, 2679 bp, Artificial Sequence
  The first nucleic acid fragment
aaataactcgagctgcgggcgctgttgttccatcatcctgtcgggcatcgcaatgcgattgtgttatatcgccgtggtggccggggtggtgctcgtggcgcttcactacgagcaggagatccagaggcgcctgtttgatgtatgacgtcacatccaggccggcggaaaccggaacggcatatgcaaactggaaactgtcctgtcttggggcccacccacccgacgcgtcatatgtaaatgaaaatcgttcccccgaggccatgtgtagcctggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggtacaataaaaacaaaacatttcaaacaaatcgccccacgtgttgtccttctttgctcatggccggcggggcgtgggtcacggcagatggcgggggtgggcccggcgtacggcctgggtgggcggagggaactaacccaacgtataaatccgtccccgctccaaggccggtgtcatagtgcccttaggagcttcccgcccgggcgcatccccccttttgcactatgacagcgacccccctcaccaacctgttcttacgggccccggacataacccacgtggcccccccttactgcctcaacgccacctggcaggccgaaacggccatgcacaccagcaaaacggactccgcttgcgtggccgtgcggagttacctggtccgcgcctcctgtgagaccagcggcacaatccactgctttttctttgcggtatacaaggacacccaccatacccctccgctgattaccgagctccgcaactttgcggacctggttaaccacccgccggtcctacgcgaactggtcgaagctttttata

SEQ ID NO: 2 - DNA, 3285 bp, Artificial Sequence
  The second nucleic acid fragment
ctcgagctgcgggcgctgttgttccatcatcctgtcgggcatcgcaatgcgattgtgttatatcgccgtggtggccggggtggtgctcgtggcgcttcactacgagcaggagatccagaggcgcctgtttgatgtatgacgtcacatccaggccggcggaaaccggaacggcatatgcaaactggaaactgtcctgtcttggggcccacccacccgacgcgtcatatgtaaatgaaaatcgttcccccgaggccatgtgtagcctggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggccttgacctttgctttactggtggccctcctggtgctcagctgcaagtcaagctgctctgtgggcttagaagttctttttcaaggtcctgcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttagtgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtcctggccggaccacgctatctgtgcaaggtccccggccccggacgcgcgctccatgagcagagcgcccgccgccgaggcgaagactcgggcggcgccctgcccgtcccaccaggtcaacaggcggtaaccggcctcttcatcgggaatgcgcgcgaccttcagcatcgccggcatgtccccctggcggacgggaagtatcaagcttgtacaataaaaacaaaacatttcaaacaaatcgccccacgtgttgtccttctttgctcatggccggcggggcgtgggtcacggcagatggcgggggtgggcccggcgtacggcctgggtgggcggagggaactaacccaacgtataaatccgtccccgctccaaggccggtgtcatagtgcccttaggagcttcccgcccgggcgcatccccccttttgcactatgacagcgacccccctcaccaacctgttcttacgggccccggacataacccacgtggcccccccttactgcctcaacgccacctggcaggccgaaacggccatgcacaccagcaaaacggactccgcttgcgtggccgtgcggagttacctggtccgcgcctcctgtgagaccagcggcacaatccactgctttttctttgcggtatacaaggacacccaccatacccctccgctgattaccgagctccgcaactttgcggacctggttaaccacccgccggtcctacgcgaactgctcgag

SEQ ID NO: 3 - DNA, 1550 bp, Artificial Sequence
  The third nucleic acid fragment
aagcttgaattcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgtaggcgtgtacggtgggaggcctatataagcagagctcgtttagtgaaccgtcagatcgcctggagccaccatgggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaatagtaatgatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtcctggccggaccacgctatctgtgcaaggtccccggccccggacgcgcgctccatgagcagagcgcccgccgccgaggcgaagactcgggcggcgccctgcccgtcccaccaggtcaacaggcggtaaccggcctcttcatcgggaatgcgcgcgaccttcagcatcgccggcatgtccccctggcggacgggaagtatcaagctt

SEQ ID NO: 4 - DNA, 426 bp, Artificial Sequence
  The nucleotide sequence of the telomerase reverse transcriptase promoter
gaattcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctg

SEQ ID NO: 5 - protein, 182 aa, Artificial Sequence
  The amino acid sequence of human rhinovirus 3C protease
GPNTEFALSLLRKNIMTITTSKGEFTGLGIHDRVCVIPTHAQPGDDVLVNGQKIRVKDKYKLVDPENINLELTVLTLDRNEKFRDIRGFISEDLEGVDATLVVHSNNFTNTILEVGPVTMAGLINLSSTPTNRMIRYDYATKTGQCGGVLCATGKIFGIHVGGNGRQGFSAQLKKQYFVEKQ

SEQ ID NO: 6 - DNA, 552 bp, Artificial Sequence
  The nucleotide sequence of the open reading frame of human rhinovirus 3C protease
atgggaccaaacacagaatttgcactatccctgttaaggaaaaacataatgactataacaacctcaaagggagagttcacagggttaggcatacatgatcgtgtctgtgtgatacccacacacgcacagcctggtgatgatgtactagtgaatggtcagaaaattagagttaaggataagtacaaattagtagatccagagaacattaatctagagcttacagtgttgactttagatagaaatgaaaaattcagagatatcaggggatttatatcagaagatctagaaggtgtggatgccactttggtagtacattcaaataactttaccaacactatcttagaagttggccctgtaacaatggcaggacttattaatttgagtagcacccccactaacagaatgattcgttatgattatgcaacaaaaactgggcagtgtggaggtgtgctgtgtgctactggtaagatctttggtattcatgttggcggtaatggaagacaaggattttcagctcaacttaaaaaacaatattttgtagagaaacaatag

SEQ ID NO: 7 - protein, 23 aa, Artificial Sequence
  The amino acid sequence of the interferon A2 signal peptide
MALTFALLVALLVLSCKSSCSVG

SEQ ID NO: 8 - DNA, 69 bp, Artificial Sequence
  The second nucleic acid encoding the interferon A2 signal peptide
atggccttgacctttgctttactggtggccctcctggtgctcagctgcaagtcaagctgctctgtgggc

SEQ ID NO: 9 - DNA, 24 bp, Artificial Sequence
  The nucleotide sequence of the third nucleic acid sequence
ttagaagttctttttcaaggtcct

SEQ ID NO: 10 - DNA, 66 bp, Artificial Sequence
  The base sequence of the CMV enhancer
gtaggcgtgtacggtgggaggcctatataagcagagctcgtttagtgaaccgtcagatcgcctgga